(19) United States
(12) Patent Application Publication
(10) Pub. No.: US 2018/0361111 A1
(43) Pub. Date: Dec. 20, 2018

(54) 3D CENTESIS DRAINAGE CATHETER DEVICE

(71) Applicant: H. LEE MOFFITT CANCER CENTER AND RESEARCH INSTITUTE, INC., Tampa, FL (US)

(72) Inventor: Bela Kis, Tampa, FL (US)

(21) Appl. No.: 16/062,125

(22) PCT Filed: Dec. 13, 2016
(86) PCT No.: PCT/US16/66290

§ 371 Date: Jun. 14, 2018

Related U.S. Application Data
(60) Provisional application No. 62/267,043, filed on Dec. 14, 2015.

(51) Int. Cl. A61M 25/00; A61M 1/00; A61M 25/01

(52) U.S. Cl. CPC A61M 25/007; A61M 27/00; A61M 25/0102; A61M 1/008

(57) ABSTRACT

Various implementations of a drainage catheter device include an outer drainage catheter and a distal portion. The outer drainage catheter has a proximal end and a distal end. The distal portion is adjacent the distal end of the outer drainage catheter and extends straight in a first configuration and forms a plurality of loops in a second configuration. A surface of the distal portion that faces radially inward in the second configuration defines a plurality of openings.

### BACKGROUND

Centesis drainage catheters are used to percutaneously drain fluid collections within the body and they are removed from the body after the drainage procedure. Traditional centesis drainage catheters are flexible plastic catheters with inner hollow stylet metal needles. The drainage catheter-metal needle assembly is inserted through the skin and tissues into the fluid collection. The inner hollow stylet metal needle permits aspiration of fluid via the needle confirming the proper catheter position in the fluid collection before the needle removal. The available centesis drainage catheters have an endhole and a few side holes close to the distal tip of the catheter. Most of the traditional centesis drainage catheters are straight or have one distal loop. When attempting to drain a fluid collection through these traditional drainage catheters, body structures, such as organs, membranes, large debris in the fluid collection, and surrounding tissues, are sucked towards the catheter and often obstruct the holes of the catheter, blocking the fluid drainage which requires catheter repositioning or abortion of the drainage procedure with incomplete drainage of the fluid.

### BRIEF SUMMARY

Various implementations include a drainage catheter device that includes an outer drainage catheter and a distal portion. The outer drainage catheter has a proximal end and a distal end. The distal portion is adjacent the distal end of the outer drainage catheter and extends straight in a first configuration and forms a plurality of loops in a second configuration. A surface of the distal portion that faces radially inward in the second configuration defines a plurality of openings.

In some implementations, the device further includes a hollow stylet metal needle that is disposable within the outer drainage catheter. The hollow stylet metal needle is extendable axially through the outer drainage catheter between the proximal end and the distal end to urge the distal portion into the first configuration. And, the hollow stylet metal needle is removable from the outer drainage catheter to allow the distal portion to form the loops of the second configuration.

In some implementations, the distal end defines an opening.

In some implementations, the loops include at least 2.5 loops.

In some implementations, the openings defined on the radially inward surface of the loops are evenly spaced apart. For example, in some implementations, the openings defined on the radially inward surface of the loops are spaced apart by a quarter turn of each loop. In addition, in certain implementations having openings spaced apart by a quarter turn of each loop, an area of the radially inward surface of the loops that defines the openings has a first end and a second end, and the first end is spaced apart distally from a proximal end of the distal portion by a half loop, and the second end is spaced apart proximally from the distal end of the outer drainage catheter by a half loop.

In some implementations, a length of the distal portion is about 7.85 centimeters in the first configuration. And, in some implementations, a length of the outer drainage catheter between the proximal end and the distal end in the first configuration is between about 14 centimeters and about 18 centimeters.

In some implementations, the device further includes a hub coupled to the proximal end of the outer drainage catheter.

In some implementations, the loops have a diameter of about 1 centimeters.

In some implementations, the outer drainage catheter comprises a plastic material.

### BRIEF DESCRIPTION OF THE DRAWINGS

These and other features, aspects, and advantages of the present invention will become apparent from the following description and the accompanying exemplary implementations shown in the drawings, which are briefly described below.

FIG. 1 illustrates a side view of a drainage catheter device in a straight configuration according to one implementation.

FIG. 2A illustrates a side view of an outer drainage catheter of the drainage catheter device of FIG. 1 in a looped configuration.

FIG. 2B illustrates a front view of the outer drainage catheter of FIG. 2A.

FIG. 2C illustrates a side view of a needle removed from the outer drainage catheter.

FIG. 2D illustrates a perspective view of the outer drainage catheter of FIG. 2A.

### DETAILED DESCRIPTION

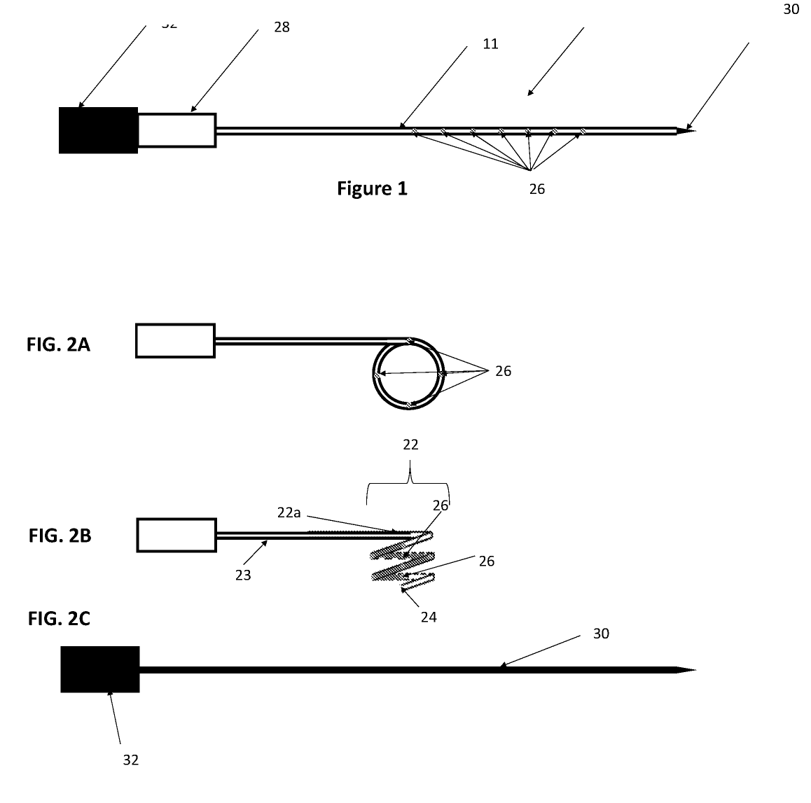

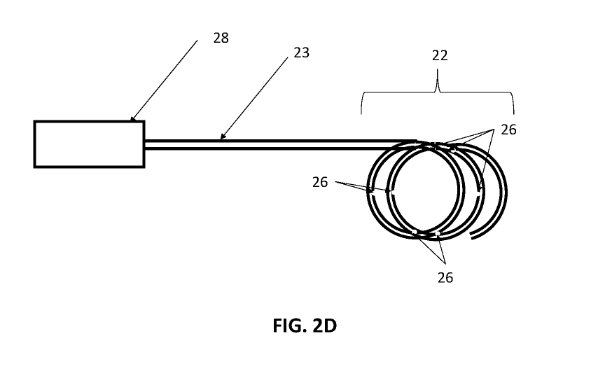

Various implementations of a 3D centesis drainage catheter are designed to achieve faster and more complete evacuation of various fluid collections from the body, including ascites, pleural effusion, cysts, and abscesses amongst others. For example, the implementation shown in FIGS. 1 and 2A-2D of the drainage catheter device 10 comprises an outer drainage catheter 20 that defines side holes 26 and an inner hollow stylet metal needle 30. The assembled device 10 has a straight configuration when the rigid inner needle 30 is inserted within the outer drainage catheter 20, which is shown in FIG. 1. When the metal needle 30 is removed, the outer drainage catheter 20 forms a looped configuration, as shown in FIGS. 2A, 2B, and 2D.

In use, the assembled drainage catheter device 10 is inserted through the skin and tissues into a fluid collection area. The inner hollow stylet metal needle 30 permits aspiration of fluid via the needle 30, confirming the proper catheter position in the fluid collection area before the needle 30 is removed. When the device 10 reaches the fluid collection area signaled by fluid aspiration via the needle 30, a hub 32 of the needle is held in place and the outer drainage catheter 20 is advanced into the fluid collection area. The outer drainage catheter 20 includes a hub 28 that may be used to advance the catheter 20. Then, the needle 30 is removed from the outer drainage catheter 20.

As is shown in FIGS. 2A, 2B, and 2D, following the removal of the inner metal needle 30, a distal portion 22 of the outer drainage catheter 20 forms a plurality of loops (turns) and conforms into a 3D structure. This 3D structural arrangement of the distal portion 22 of the outer catheter 20 keeps away tissues, membranes, and other structures, which potentially obstructs sideholes 26 defined by the drainage catheter 20. For example, the drainage catheter 20 forms 2.5 loops, and the sideholes 26 are positioned 90 degrees from each other on the surface of the loops facing radially inwardly (toward the center of each loop). The sideholes 26 start at a proximal half loop and continue for 1.5 loops. This means that the 1.5 loops with 7 sideholes 26 are protected by half loops on each end of the distal portion 22. Having 2.5 loops, or turns, provides sufficient surface area and occupies a sufficient amount of three dimensional space within the fluid filled cavity to keep organs, tissues, membranes and other body parts away from the sideholes in the loops. The sideholes 26 may be evenly spaced apart, according to some implementations. For example, the sideholes 26 are spaced apart about 0.785 cm for a loop having a diameter of about 1 cm.

The drainage catheter 20 may be plastic or other self-deformable material with “inner memory” to form the loops following the needle 30 removal.

However, in other implementations, the distal portion 22 may define more than one loop in the looped configuration and/or may define two or more sideholes, or openings, that are spaced apart in any suitable configuration, depending on the dimensions of the distal portion, the diameter of each loop, and/or the intended use of the drainage catheter device.

The dimensions of various portions of the outer drainage catheter 20 may be selected depending on the intended area to be drained by the device 10 and/or the size of the patient. For example, a length of the distal portion 22 of the outer drainage catheter 20 may be about 7.85 centimeters, and a length of a stem portion 23 of the outer drainage catheter 20, which extends between the proximal end of the device 10 and a proximal end 22a of the distal portion 22, is between about 6 centimeters and about 10 centimeters. For example, the stem 23 may be 6 cm long for average sized patients and 10 cm long for larger patients. Thus, a total “working” length of the outer drainage catheter 20 may be about 14 to about 18 centimeters in the straight configuration. In addition, the catheter hub 28 may have a length of about 1.5 centimeters. The loops of the distal portion 22 may have a diameter of about 1 centimeter when in the looped configuration. However, in other implementations, the diameter of the loops may be increased or decreased, depending on the intended use of the device 10 and/or the materials used for the device 10, which may change the total working length of the outer drainage catheter 20.

According to various implementations, the 3D centesis drainage catheter device, such as device 10 described above, eliminates many of the problems that traditional drainage catheters have during regular use. For example, intracavitary structures (such as the pleura and lungs in the chest and the omentum and bowel loops in the abdomen) often stick to the openings in the traditional drainage catheters due to the suction of the catheter, which obstructs the flow of the drainage fluid. The 3D centesis drainage catheter device according to various implementations avoids this problem by physically pushing away these intracavitary structures with the outer radial surfaces of the loops. In addition, defining the openings on the inner radial surface of the loops ensures that the intracavitary structures are not sucked towards the outer drainage catheter and obstruct the flow of drainage fluid through the openings and into the outer drainage catheter.

The corresponding structures, materials, acts, and equivalents of all means or step plus function elements in the claims below are intended to include any structure, material, or act for performing the function in combination with other claimed elements as specifically claimed. The descriptions of various implementations of the present invention have been presented for purposes of illustration and description, but are not intended to be exhaustive or limited to the invention in the form disclosed. Many modifications and variations will be apparent to those of ordinary skill in the art without departing from the scope and spirit of the invention. The implementations were chosen and described in order to best explain the principles of the invention and the practical application, and to enable others of ordinary skill in the art to understand the invention for various implementations with various modifications as are suited to the particular use contemplated.

1. A drainage catheter device comprising an outer drainage catheter having a proximal end, a distal end, and a distal portion adjacent the distal end, wherein the distal portion extends straight in a first configuration and forms a plurality of loops in a second configuration, and a surface of the distal portion that faces radially inward in the second configuration defines a plurality of openings.

2. The drainage catheter device of claim 1, further comprising a hollow stylet metal needle that is disposable within the outer drainage catheter, wherein the hollow stylet metal needle is extendable axially through the outer drainage catheter between the proximal end and the distal end to urge the distal portion into the first configuration, and the hollow stylet metal needle is removable from the outer drainage catheter to allow the distal portion to form the loops of the second configuration.

3. The drainage catheter device of claim 1, wherein the distal end defines an opening.

4. The drainage catheter device of claim 1, wherein the loops comprise at least 2.5 loops.

5. The drainage catheter device of claim 1, wherein the openings defined on the radially inward surface of the loops are evenly spaced apart.

6. The drainage catheter device of claim 5, wherein the openings defined on the radially inward surface of the loops are spaced apart by a quarter turn of each loop.

7. The drainage catheter device of claim 5, wherein an area of the radially inward surface of the loops that defines the openings has a first end and a second end, the first end being spaced apart distally from a proximal end of the distal portion by a half loop, and the second end being spaced apart proximally from the distal end of the outer drainage catheter by a half loop.

8. The drainage catheter device of claim 1, wherein a length of the distal portion is about 7.85 centimeters in the first configuration.

9. The drainage catheter device of claim 1, wherein a length of the outer drainage catheter between the proximal end and the distal end in the first configuration is between about 14 centimeters and about 18 centimeters.

10. The drainage catheter device of claim 1, further comprising a hub coupled to the proximal end of the outer drainage catheter.

11. The drainage catheter device of claim 1, wherein the loops have a diameter of about 1 centimeters.

12. The drainage catheter device of claim 1, wherein the outer drainage catheter comprises a plastic material.
